(19) United States
(12) Plant Patent Application Publication
(10) Pub. No.: US 2004/0006801 P1
(43) Pub. Date: Jan. 8, 2004

(54) Tree lilac plant named 'Golden Eclipse'

(76) Inventor: Robert Bakker, St. Catharines (CA)

Correspondence Address: BURNS DOANE SWECKER & MATHIS L L P, POST OFFICE BOX 1404, ALEXANDRIA, VA, 22313-1404, US

(21) Appl. No.: 10/420,824

(22) Filed: Apr. 23, 2003

(30) Foreign Application Priority Data
Feb. 26, 2001 (CA) 01-2536

(51) Int. Cl. A01H 5/00

(52) U.S. Cl. PLT/248

(57) ABSTRACT

A new and distinct highly ornamental cultivar of Syringa reticulata Japanese Tree Lilac is provided. The Lilac plant has an upright compact growth habit. The foliage possesses variegation that initially is green with an irregular darker green center wherein the margins turn to a stable bright yellow-gold coloration upon maturity. The blossoms are white and are displayed during late spring to early summer. Good winter hardiness is made possible and the plant has been found to be free of insect and disease problems. The plant is particularly wellsuited for providing distinctive ornamentation in the landscape.

### SUMMARY OF THE INVENTION

The new Lilac plant of the present invention was discovered during 1995 in a nursery setting at St. Catharines, Ontario, Canada, while growing within a large block of Syringa reticulata seedlings (non-patented in the United States). The new variety was primarily selected because of the distinctive appearance of the foliage and is believed to be a whole plant mutation of unknown causation. Had the new variety not been discovered and preserved, it would have been lost to mankind.

It was found that the cultivar of Syringa reticulata exhibits the following combination of characteristics:

(a) exhibits an upright compact growth habit,

(b) forms attractive variegated foliage that initially is green with an irregular darker green center wherein the margins turn to a stable bright yellow-gold coloration upon maturity,

(c) forms fragrant white flowers in late spring to early summer,

(d) is relatively free of insect and disease problems, and

(e) is well-suited for providing distinctive ornamentation in the landscape.

The stability of the variegated foliage has been well confirmed during observations to date. Such coloration also has displayed excellent resistance to sunburn.

The new cultivar of the present invention can be readily distinguished from the ‘Ivory Silk’ cultivar (non-patented in the United States). For instance, the ‘Ivory Silk’ cultivar lacks variegated foliage, displays less anthocyanin coloration on one year-old shoots, and forms larger flowers.

The new cultivar of the present invention has been asexually propagated beginning in 1996 and each year thereafter at St. Catharines, Ontario, Canada, by summer budding on Syringa reticulata rootstock. The distinctive characteristics of the new cultivar including the variegated appearance of the foliage have been found to be stable and to be capable of transmission from one generation to another following such asexual propagation.

The new cultivar of the present invention has been named ‘Golden Eclipse’.

### BRIEF DESCRIPTION OF THE PHOTOGRAPHS

The accompanying photographs show, as true as is reasonably possible to make the same in color illustrations of this character, the distinctive foliage of the new cultivar. The plants were propagated by sumemr budding on Syringa reticulata understock and were growing in the field at St. Paul, Minn., U.S.A.

FIG. 1—shows the new green foliage as it emerges in the springtime. The darker green irregular central area of the leaves is apparent. The illustrated plant was approximately three years of age.

FIG. 2—shows a closer view of typical leaves during the summer. The margins of the leaves have turned to bright yellow-gold, and the irregular dark green area in the center of the leaves gives the illusion of an eclipse. The illustrated plant was approximately three years of age and was tied to a pole for added support during the early years of its growth. As the tree matures, such support can be removed.

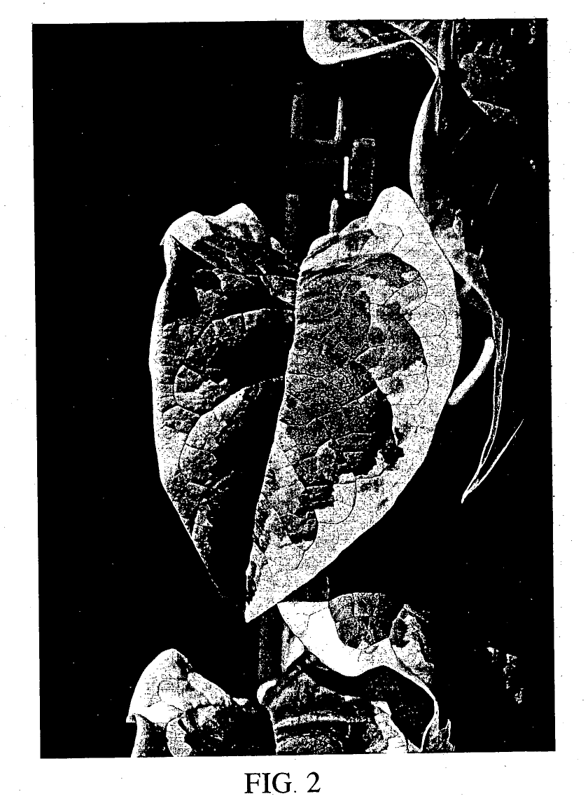

FIG. 3—shows the top portion of a typical tree bearing the distinctive variegated foliage during the summer. The tree displays a straight trunk and short branches, and was approximately two years of age.

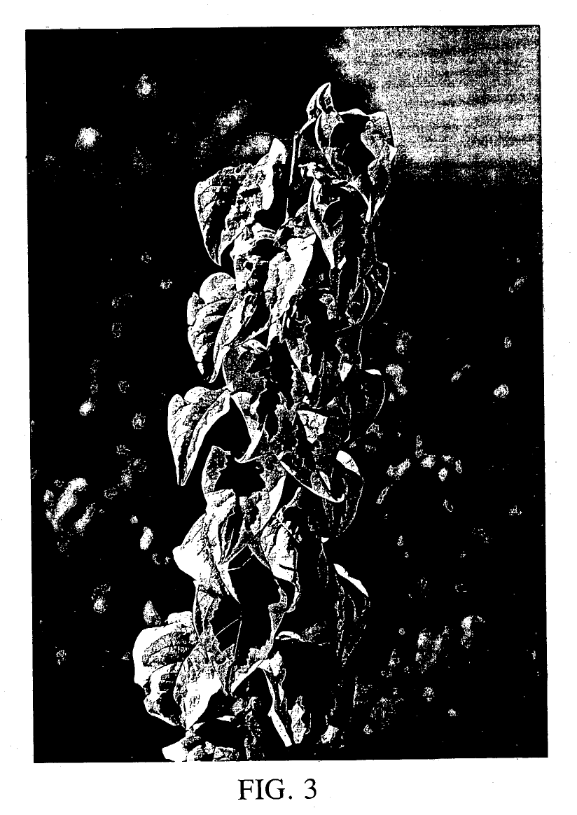

### DETAILED DESCRIPTION

The chart used in the identification of colors is the R.H.S. Colour Chart of The Royal Horticultural Society, London, England. Common terms are to be accorded their ordinary dictionary significance. The description is based upon the observation of two and three year-old plants that were summer budded on Syringa reticulata understock and were growing in the field at St. Catharines, Ontario, Canada.

Botanical classification: Syringa reticulata, cv. ‘Golden Eclipse’.

Plant:

Growth habit.—Upright, compact, and bushy.

Size.—A one year-old tree commonly displays a height of approximately 4 to 5 feet, a two year-old tree commonly displays a height of approximately 6 to 8 feet, and a three year-old tree commonly displays a height of approximately 7 to 10 feet. A ten to fifteen year-old tree is anticipated to assume a height of approximately 18 to 24 feet and a spread of approximately 8 to 14 feet.

Density of crown.—Medium.

Growth rate.—Similar to other Lilacs.

Stem characteristics:

Color.—On a young shoot the coloration changes from green to olive-brown with anthocyanin coloration of medium intensity. Such anthocyanin coloration is considerably stronger than that of the ‘Ivory Silk’ cultivar.

Lenticels.—Medium in number on a one year-old shoots on the sunny side.

Pubescence.—Very sparse on a one year-old shoot.

Vegetative bud.—Bears no or very sparse pubescence.

Bark:

Color.—On a one year-old shoot the bark is reddish-brown on the sunny side. This compares to a greenish-grey coloration for the ‘Ivory Silk’ cultivar.

Foliage:

Leaf shape.—Ovate.

Form.—Simple.

Leaf apex.—Acute.

Leaf base.—Rounded.

Leaf margins.—Entire with weak undulation.

Cross-section.—Slightly convex.

Pubescence.—None.

Upper surface.—Medium glossiness.

Glaucosity.—Absent.

Texture.—Leathery.

Length.—Approximately 18 to 21 cm (mean 19 cm). This can be compared to approximately 15 to 18 cm (mean 17 cm) for the ‘Ivory Silk’ cultivar. The leaves tend to be larger on very young trees.

Width.—Approximately 11 to 12 cm (mean 11.5 cm). This can be compared to approximately 12 cm for the ‘Ivory Silk’ cultivar. The leaves tend to be larger on very young trees.

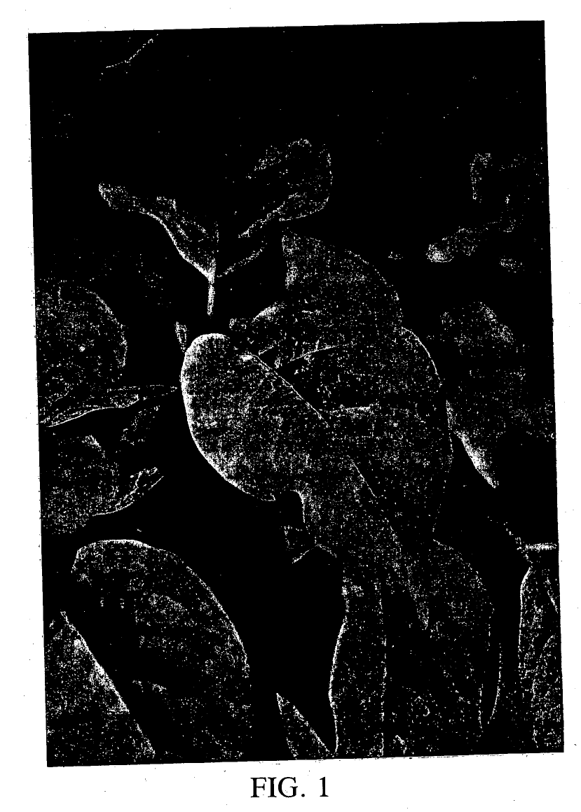

Color.—The leaf blade is variegated while that of the ‘Ivory Silk’ cultivar has no variegation. As shown in FIG. 1 initially the leaves are green with an irregular darker green central area. As the leaves mature during the summer, the variegated appearance becomes more apparent with the leaf margins assuming a bright yellow-gold appearance and the irregular dark green central area gives an illusion of an eclipse. The variegation can be seen through the underside of a leaf. The foliage has been found to hold its coloration well even in full sun. Also, the foliage well resists sunburn. The fall foliage coloration is insignificant.

Petioles.—Commonly approximately 1.25 cm in length.

Inflorescence:

Arrangement.—In semi-erect panicles and mainly from terminal buds as the ‘Ivory Silk’ cultivar.

Buds.—Yellow-White Group 158C in coloration when unopened. This compares to Yellow-White Group 158D for the ‘Ivory Silk’ cultivar.

Flower configuration.—Very short funnel-shaped corolla tubes with four flat to reflexed rounded corolla lobes. The lobes are flat to reflexed in cross section when the flower is open.

Flower size.—Approximately 4 to 6 mm (5 mm mean). This compares to approximately 7.9 mm (8 mm mean) for the ‘Ivory Silk’ cultivar.

Flower color.—When ⅔ open, the coloration is White Group 155B. This can be compared to White Group 155D when ⅔ open for the ‘Ivory Silk’ cultivar. When fully open, the upper side of the corolla lobes is White Group 155D. This can be compared to lighter than White Group 155D for the ‘Ivory Silk’ cultivar. The outer surface of the corolla tube is Yellow-White Group 158D.

Stamens.—Approximately the same length as the corolla tube.

Anthers.—White in coloration.

Fragrance.—Medium intensity.

Flowering time.—Late spring to early summer, and commenced on Jun. 20, 2002. Flowers commonly are absent on one and two year-old trees.

Hardiness: Can be grown in U.S.D.A. Hardiness Zone No. 4.

Culture: Similar to the ‘Ivory Silk’ cultivar.

Vegetation: Vigorous.

Disease/Pest/Resistance: Has proven to be very disease and pest resistant during observations to date. No pesticide applications have been needed.

Landscape usage: Provides a hardy highly distinctive fragrant tree having a distinctive foliage coloration that can be grown as a specimen plant to provide attractive ornamentation in the landscape.

I claim:

1. A new and distinct Syringa reticulata plant having the following combination of characteristics:
(a) exhibits upright compact growth habit,
(b) forms attractive variegated foliage that initially is green with an irregular darker green center wherein the margins turn to a stable bright yellow-gold coloration upon maturity,
(c) forms fragrant white flowers in the late spring to early summer,
(d) is relatively free of insect and disease problems, and
(e) is well-suited for providing distinctive ornamentation in the landscape;
substantially as illustrated and described.
